(19) United States
(12) Patent Application Publication
(10) Pub. No.: US 2021/0307908 A1
(43) Pub. Date: Oct. 7, 2021

(54) STRENGTH ENHANCED ADDITIVELY MANUFACTURED MEDICAL IMPLANT AND METHODS

(71) Applicant: Smith & Nephew, Inc., Memphis, TN (US)

(72) Inventor: Angela Mines, Arlington, TN (US)

(21) Appl. No.: 17/270,193

(22) PCT Filed: Aug. 21, 2019
(86) PCT No.: PCT/US2019/047500

§ 371 Date: Feb. 22, 2021

Related U.S. Application Data
(60) Provisional application No. 62/724,375, filed on Aug. 29, 2018.

(51) Int. Cl. A61F 2/30; B33Y 10/00; B33Y 70/00; B33Y 80/00; B29C 64/188; B29C 64/153; B22F 10/20; B22F 5/10; A61F 2/38

(52) U.S. Cl. CPC A61F 2/3094; A61F 2310/00017; B33Y 70/00; B33Y 80/00; B29C 64/188; B29C 64/153; B22F 10/20; B22F 5/10; A61F 2/389; A61F 2/3886; A61F 2002/30985; A61F 2002/3097; A61F 2002/30968; A61F 2310/00023; B33Y 10/00

(57) ABSTRACT

Medical implants and methods for forming medical implants with additive manufacturing techniques are provided herein. The medical implants include arranging grain orientations of the materials of the medical implants to enhance strength characteristics of the medical implants or to reduce material requirements. The medical implant may be formed by heating a first portion of a granulized material along a first build pathway to melt the material such that grains of the material are oriented along the first build pathway after the melted material cools and heating a second portion of the granulized material along a second build pathway to melt the material such that grains of the material are oriented along the second build pathway oriented substantially transverse to the build pathway of the first portion after the melted material cools. The first and second build pathways may be a series of substantially overlapping melting events.

### CROSS-REFERENCE TO RELATED APPLICATION

This application claims the benefit of U.S. Provisional Patent Application Ser. No. 62/724,375, filed Aug. 29, 2018, entitled “Strength Enhanced Additively Manufactured Medical Implant and Methods”, the entire contents of which application is hereby incorporated by reference in its entirety.

### TECHNICAL FIELD

The present invention relates generally to the field of orthopedic medical implants, and more particularly relates to methods of manufacturing, forming, etc. medical implants via an additively manufacturing technique arranged and configured to orientate the grains of the granularized material used to manufacture the medical implant so that the resulting medical implant is designed to better withstand likely loadings.

### BACKGROUND

Generally speaking, conventional medical implants such as, for example, knee implants, bone plates, intramedullary nails, etc. have uniform physical properties along all three dimensional axes of the implant because the implants are typically machined from a billet of material. That is, in a typical billet of material, grains tend to be oriented more or less randomly. Grain orientation (e.g., orientating the grain of the billet of material in a certain orientation or pathway), however, may be tailored to enhance certain physical characteristics such as bending strength and fatigue strength. For example, some additive manufacturing methods can produce grain orientations in a direction of a build pathway taken by an energy source, such as a laser, as the laser melts an additive manufacturing powder and the molten material cools.

Therefore, an improved physical structure can be produced by prescribing an improved build pathway that takes into consideration the grain orientations that result from the build pathway taken by the energy source used to additively manufacture the structure.

It is with this in mind that the present disclosure is provided.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

Disclosed herein is a method of manufacturing, forming, etc. (used interchangeably herein without the intent to limit) one or more medical implants via an additively manufacturing technique arranged and configured to orientate the grains of the granularized material (e.g., billet of material) used to manufacture the medical implant so that the resulting medical is designed to better withstand likely loadings.

In one embodiment, the method of forming the medical implant includes a build pathway that creates grain structures in locations and at orientations that improve strength and other physical characteristics of the resulting medical implant. In one embodiment, the medical implant may have increased strength per unit weight by more efficiently aligning the grain orientations of the material used to create the medical implant. In addition, and/or alternatively, additional improvements may be achieved by optimization of a medical implant to best fit a specific patient or a particular subset of patients.

In one embodiment, a method of forming an orthopedic medical implant is disclosed. The method comprising heating a first portion of a granulized material by irradiating the first portion of the granulized material along a first build pathway to melt the material such that grains of the material are oriented along the first build pathway after the melted material cools, wherein the first build pathway is a series of substantially overlapping melting events; and heating a second portion of the granulized material by irradiating the second portion of the granulized material along a second build pathway to melt the material such that grains of the material are oriented along the second build pathway after the melted material cools, wherein the second build pathway is a series of substantially overlapping melting events that are oriented at least in part transversely to the orientation prevailing direction of the build pathway of the first portion; wherein the act of heating a first portion of the granulized material by irradiating the first portion of the granulized material includes irradiating with a laser light beam such that a part of the first portion larger than a diameter of the laser light beam is molten while the first portion is being formed and adjacent areas overlap with one another primarily linearly along the build pathway.

In some embodiments, the first build pathway is a series of substantially overlapping melting events in a circular pattern.

In some embodiments, the second build pathway is a series of substantially overlapping melting events in a circular pattern.

In some embodiments, heating a first portion of a granulized material is performed by application of consistent heat and speed to the first portion of the granulized material.

In some embodiments, heating the first portion and second portion is performed by application variable heat to the first and second portions, respectively.

In some embodiments, the application of variable heat to the first and second portions is sufficient to polarize the material along the first build pathway of sufficient gradient depth and width to polarize the material.

In some embodiments, the medical implant is a knee arthroplasty implant including a tibial component including a tibial plateau and a stem portion, the first portion comprising the tibial plateau, the second portion comprising the stem portion.

In some embodiments, the tibial plateau further comprises a perimeter portion, the perimeter portion being formed by: heating the perimeter portion of the granulized material by irradiating the perimeter portion of along a perimeter build pathway to melt the material such that grains of the material are oriented along the perimeter build pathway after the melted material cools, wherein the perimeter build pathway is a series of substantially overlapping melting events that are oriented at least in part transversely to the orientation prevailing direction of the build pathway of the tibial pathway.

In some embodiments, the medical implant includes a body portion having one or more openings formed therein, the first portion comprising the body portion, the second portion comprising an area surrounding the one or more openings.

In some embodiments, the medical implant is a bone plate including a body and one or more openings formed therein, the first portion comprising the body of the bone plate, the second portion comprising an area surrounding the one or more openings.

In some embodiments, the body of the bone plate includes a longitudinal axis, the first build pathway being orientated along the longitudinal axis of the bone plate.

In some embodiments, the medical implant is an intramedullary nail including a body and one or more openings formed therein, the first portion comprising the body of the intramedullary nail, the second portion comprising an area surrounding the one or more openings.

In some embodiments, the body of the intramedullary nail includes a longitudinal axis, the first build pathway being orientated along the longitudinal axis of the intramedullary nail.

In some embodiments, the granulized material is selected from one of a titanium alloy, a steel alloy, a metal, a polymer, or any effective combination of granulized materials.

In another embodiment, the resulting medical implant includes a first portion additively manufactured by melting a granulized material along a build plane of the first portion with a series of substantially overlapping melting events to orient a first set of grains of the material after cooling along the build plane of the first portion. The medical implant may also include a second portion additively manufactured at least in part to the first portion by melting the granulized material to orient a second set of grains of the material after cooling at least in substantial part transversely to the orientation of the first set of grains. The first portion may be configured for improved resistance to moment forces to be applied into the build plane of the first portion by loading of the medical device created when the medical implant is implanted and used.

Another embodiment of the invention is a method of forming a medical implant. The method may include heating a first portion of a granulized material by irradiating the first portion of the granulized material along a first build pathway to melt the material such that grains of the material are oriented along the first build pathway after the melted material cools. The first build pathway may include a series of substantially overlapping melting events. The method may also include heating a second portion of the granulized material by irradiating the second portion of the granulized material along a second build pathway to melt the material such that grains of the material are oriented along the second build pathway after the melted material cools. The second build pathway may include a series of substantially overlapping melting events that are oriented at least in substantial part transversely to the orientation prevailing direction of the build pathway of the first portion.

Still another embodiment of the invention is a knee arthroplasty implant with a femoral component and a tibial component. The tibial component may include a tibial plateau additively manufactured by melting a granulized material along a build plane of the tibial plateau with a series of substantially overlapping melting events to orient grains of the material after cooling along the build plane of the tibial plateau. The tibial component may also include a transverse portion additively manufactured at least in part to the tibial plateau by melting the granulized material to orient grains of the material after cooling in substantial part transversely to a prevailing orientation of the build pathway along the build plane of the tibial plateau.

Yet another embodiment of the invention is a method of forming a medical implant with one or more openings. The method may include providing a granulized material and heating the granulized material by irradiating the granulized material along a build pathway. The build pathway may be configured to divert around at least one of the one or more openings to guide grain orientations around the at least one of the one or more openings without terminating at the at least one of the one or more openings.

Embodiments of the present disclosure provide numerous advantages. For example, by orientating the grain orientation of the material used to manufacture the medical implant, the resulting medical implant includes increased bending strength and fatigue strength in the direction of the expected applied loads.

Further features and advantages of at least some of the embodiments of the present invention, as well as the structure and operation of various embodiments of the present invention, are described in detail below with reference to the accompanying drawings.

### BRIEF DESCRIPTION OF THE DRAWINGS

By way of example, a specific embodiment of the disclosed device will now be described, with reference to the accompanying drawings, in which:

FIG. 1 is a perspective view of an example embodiment of a total knee arthroplasty system;

FIG. 2 is a side elevation view of the total knee arthroplasty system illustrated in FIG. 1;

FIG. 3 is a cross-sectional rear elevation view through the total knee arthroplasty system illustrated in FIG. 2;

FIG. 4 is a plan view illustrating a build pathway taken by an energy source being used to additively manufacture by successively heating a series of melt areas in accordance with one of the principles of the present disclosure;

FIG. 4A is a detail view of one of the melt areas of FIG. 4 illustrating an embodiment where four substantially overlapping melting events have been applied to form one melt area;

FIG. 5 is a front elevation view of a part of a tibial component of the total knee arthroplasty system of FIG. 1;

FIG. 6 is a cross-sectional plan view through a stem of the part of the tibial component illustrated in FIG. 5;

FIG. 6A is a detail view illustrating build pathways within the stem illustrated in FIG. 6 in accordance with one of the principles of the present disclosure;

FIG. 7 is a perspective view of the part of a tibial component illustrated in FIG. 5;

FIG. 7A is a detail view illustrating build pathways along a tibial plateau of the part of the tibial component illustrated in FIG. 7 in accordance with one aspect of the present disclosure;

FIG. 8 is a perspective view of a bone plate illustrating build pathways along the bone plate that divert around openings through the bone plate in accordance with one aspect of the present disclosure;

FIG. 9 is a side elevation view of an intramedullary nail; and

FIG. 9A is a detail view illustrating build pathways along the intramedullary nail illustrated in FIG. 9 that divert around opening through the intramedullary nail in accordance with one aspect of the present disclosure.

It should be understood that the drawings are not necessarily to scale and that the disclosed embodiments are sometimes illustrated diagrammatically and in partial views. In certain instances, details which are not necessary for an understanding of the disclosed methods and devices or which render other details difficult to perceive may have been omitted. It should be further understood that this disclosure is not limited to the particular embodiments illustrated herein.

### DETAILED DESCRIPTION

Various features, aspects, steps, or the like of methods for manufacturing, forming, etc. an orthopedic medical implant, and the resulting medical implant, will now be described more fully hereinafter with reference to the accompanying drawings, in which one or more aspects, features, steps, or the like of the method and/or resulting medical implant will be shown and described. It should be appreciated that the various features, aspects, steps, or the like may be used independently of, or in combination, with each other. It will be appreciated that the medical implants as disclosed herein may be embodied in many different forms and should not be construed as being limited to the embodiments set forth herein. Rather, these embodiments are provided so that this disclosure will convey certain aspects or features of the resulting medical implant to those skilled in the art. In the drawings, like numbers refer to like elements throughout unless otherwise noted.

Disclosed herein are improved methods, steps, etc. of manufacturing, forming, etc. (used interchangeably herein without the intent to limit) an orthopedic medical implant and corresponding resulting orthopedic medical implants. As will be described in greater detail, the method includes heating portions of a granulized material by sintering, irradiating, etc. the portion of the granulized material along a build pathway to melt the material such that grains of the material are oriented along the build pathway after the melted material cools. In one embodiment, the build pathway may include a series of substantially overlapping melting events. In addition, and/or alternatively, the medical implant may include first and second portions, each formed by heating portions of the granulized material along respective build pathways. In one embodiment, the build pathway of the second portion may be oriented at least in part transversely to the orientation of the build pathway of the first portion.

As will be described and illustrated herein, the resulting orthopedic medical implant may be in the form of a total knee arthroplasty system, a bone plate, an intramedullary nail, etc. It is envisioned that the disclosed methods may be used to manufacture any suitable medical implant and thus the medical implant should not be limited to those illustrated and described herein unless specifically claimed.

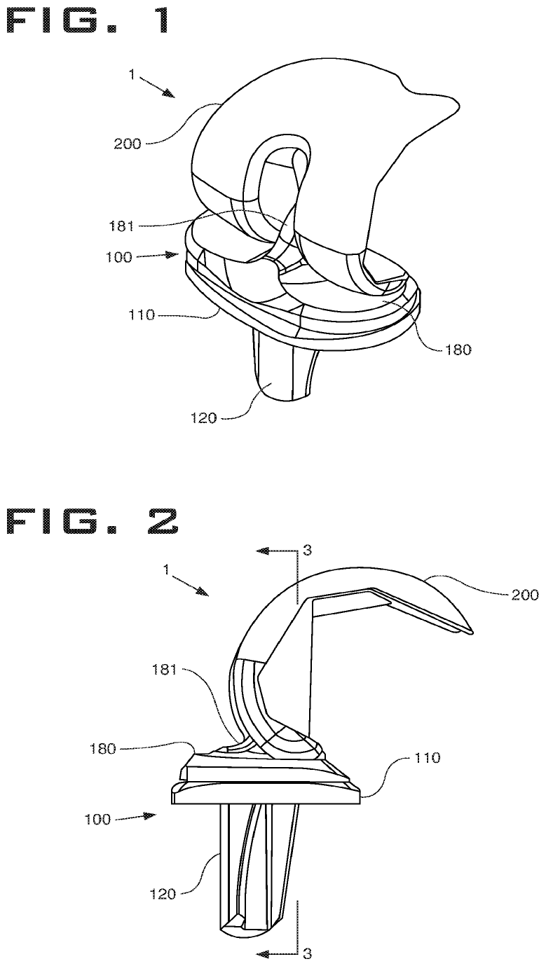

Referring to FIGS. 1-3, an example embodiment of a medical implant is disclosed. As shown, the medical implant is in the form of a total knee arthroplasty system 1 including a tibial component 100 and a femoral component 200. Some embodiments of the total knee arthroplasty system 1 may also include a patellar implant (not shown). As shown, the tibial component 100 may include a tibial plateau 110, a stem 120, and an insert 180. In addition, as shown, the total knee arthroplasty system 1 may be in the form of a metal-on-polyethylene, posterior stabilized system, as evidenced by incorporation of a post 181, which may be part of the insert 180. In use, the insert 180 may provide for a bearing, friction reduction, and spacing between the tibial plateau 110 and the femoral component 200. While the present disclosure will be described and illustrated in connection with the total knee arthroplasty system 1 of FIG. 1, it should be appreciated that the medical implant/knee system may have any suitable shaped, configuration, etc. For example, the medical implant may only include a tibial component, a femoral component, or have some other shaped or configuration, for example, a partial knee system. As such, it is envisioned that any other effective type of knee arthroplasty system, included but not limited to, cruciate retaining, posterior cruciate substituting, rotating platform, unicondular, etc. may be used in various embodiments, each of which may include metal-on-polyethylene, metal-on-metal, or any other effective interface, and may or may not include a separate insert.

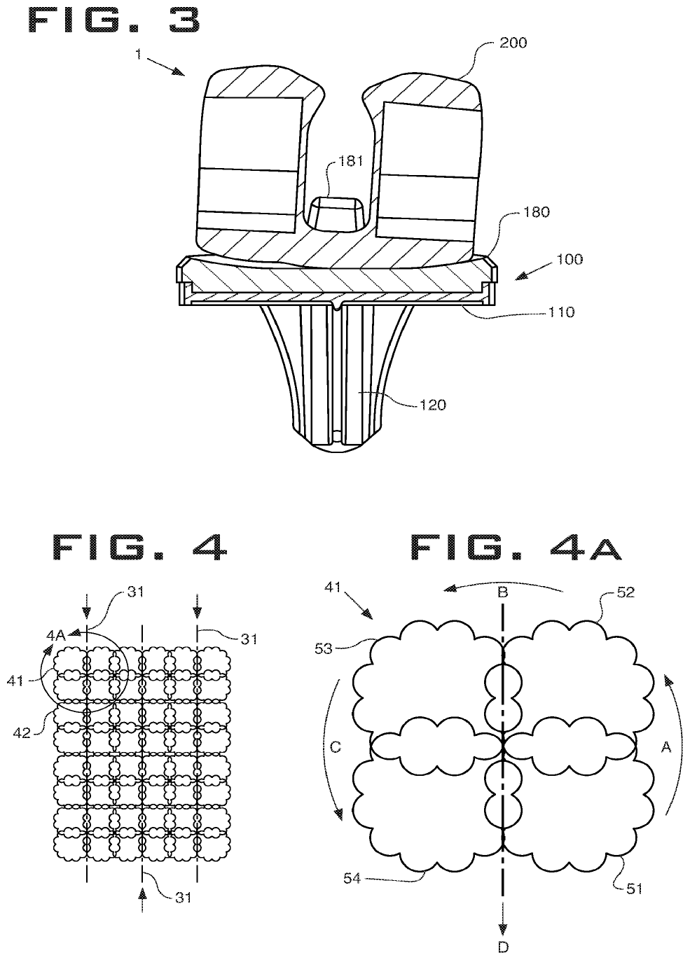

Referring to FIGS. 4 and 4A, in accordance with one or more principles of the present disclosure, the medical implant (e.g., the total knee arthroplasty system 1) may be formed with build pathways 31 and melting events 41, 42. In some embodiments, each melting event 41, 42 may be a result of multiple applications of heat or radiation, as depicted in FIG. 4A. In accordance with one embodiment of the present disclosure, heat for the melting events 41, 42 may be supplied by now known or suitable mechanism including, for example, via sintering, irradiating, etc. In this example, the melting event 41 is a result of four applications of a laser light beam at locations 51, 52, 53, 54, although it is envisioned that more or less applications may be used. The approximate diameter of these four applications of the laser light beam may be between approximately 150 microns and 1000 microns. The applications of laser light beams and melting events 41, 42 depicted are not necessarily to scale with one another in FIGS. 4 and 4A. A laser of any effective type and power may be used. In some embodiments, the effective power of the laser may be between about 100 watts and 2000 watts. The diameter of the laser light beam of some embodiments is approximately between 50 microns and 500 microns.

Referring to FIG. 4A, in accordance with one aspect of the present disclosure, the applications of the laser light beam may be applied successively and the position of the laser light beam may be moved along a build pathway generally depicted by arrows A, B, C, and D. For example, in the embodiment shown, arrow A indicates movement after application of a laser light beam at location 51, arrow B indicates movement after application of a laser light beam at location 52, arrow C indicates movement after application of a laser light beam at location 53, and arrow D indicates movement to the next melting event 42 after application of a laser light beam at location 54. The locations 51, 52, 53, 54 illustrate adjacent areas that are overlapped such that an area larger than a diameter of a laser light beam used to melt a granulized material is molten simultaneously. Further, melting event 42 may be begun while melting event 41 is still partially or completely molten such that an even larger area may be molten simultaneously. Melting event 41 and melting event 42 may be described as overlapping primarily linearly along build pathway 31. Considered on a smaller scale, heating at each location 51, 52, 53, 54 may each be considered a melting event, and in this example the melting events of adjacent areas may be described as overlapping in a substantially circular pattern. Taken together, melting at each location 51, 52, 53, 54 may be considered as melting in a substantially circular overlapped pattern and may be combined with a primarily linearly overlapped pattern from melting event 41 to melting event 42. Any other effective pattern is contemplated to be within the scope of the disclosure in other embodiments.

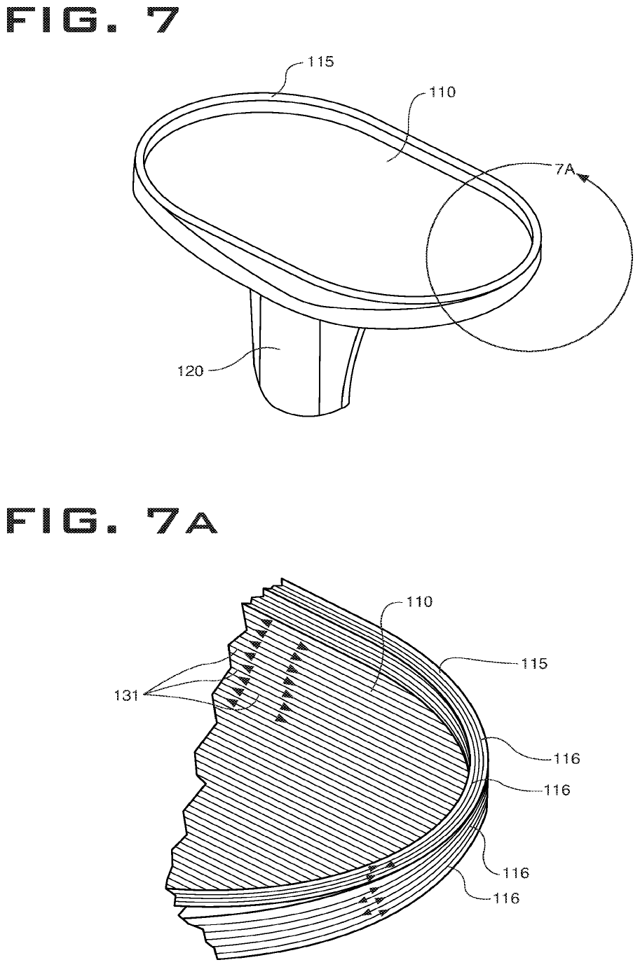

As shown in FIGS. 1-3 and 5-7A, and as previously mentioned, the medical implant may be in the form of a total knee arthroplasty system 1 including a tibial component 100. In accordance with one aspect of the present disclosure, the tibial component 100 may be formed with a first portion, embodied in a tibial plateau 110. The first portion or tibial plateau 110 may be additively manufacture by melting a granulized material along a build plane of the tibial plateau 110, as most clearly illustrated by the build pathways 131 shown in FIG. 7A. The granulized material may be any suitable material now known or hereafter developed including, for example, a titanium alloy, a steel alloy, another metal, a polymer, or any effective combination of granulized materials. The illustrated build pathways 131 are not to scale and are intended to primarily show a reciprocating direction medial to lateral and lateral to medial. For example, as will be appreciated by one of ordinary skill in the art, the laser beam light is moved across the tibial plateau 110 in a reciprocating direction medial to lateral and lateral to medial direction to form the illustrated build pathways 131. In other embodiments, build pathways of a tibial plateau could be only lateral to medial or only medial to lateral, substantially anterior to posterior, at an acute angle to a primary direction, or at a variety of angles along the build plane. In the illustrated embodiment, melting is accomplished with a series of substantially overlapping melting events as shown and described herein in association with FIGS. 4 and 4A. This act of melting via a series of substantially overlapping melting events orients a first set of grains of the melted material after cooling along the build plane of the first portion. As used herein, a grain is a portion of material within a mass with consistent crystallographic orientation bounded by a grain boundary where crystallographic orientation changes abruptly, for example, by more than about 15 degrees. Grain orientation resulting from build orientation has been shown to improve bending strength and fatigue strength of loads applied into the build plane. See W. K. de Vree., “On the influence of build orientation on the mechanical properties of direct metal laser sintered (DMLS) Ti-6Al-4V flexures”, 2016, Masters Thesis Reference: “uuid:e775454d-b4c8-4b59-b126-c6b50820lafa”, which is incorporated by reference herein in its entirety.

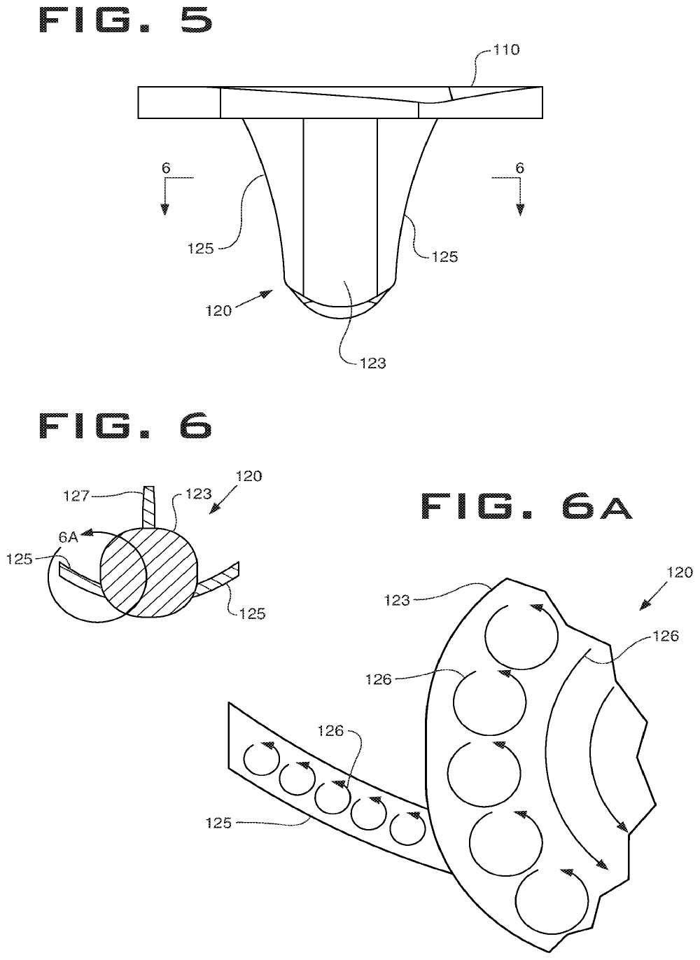

The tibial component 100 includes a second portion additively manufactured at least in part to the first portion by melting the granulized material to orient a second set of grains of the material after cooling at least in substantial part transversely to the orientation of the first set of grains. For example, in the case of the tibial component 100, the second portion may be one or both of the stem 120 and the perimeter portion 115 around the tibial plateau 110 of the tibial component 100. The second portion may also be referred to herein as a transverse portion having grains oriented transversely to the typical or prevailing orientation of the build pathway 131 along the build plane of the tibial plateau 110. As shown in FIGS. 5-6A, the stem 120 may include a stem body 123, a pair of gussets 125, and a posterior fin 127, although other configurations are envisioned. A set of successive, rounded, and closed patterned build directions (shown by curved arrows 126) used to form multiple tubes along a longitudinal axis of the stem 120 are depicted in FIG. 6A. By applying layers of rounded and closed patterned build pathways for successive additive manufacturing passes, effective tubes of grain orientations can be achieved that include grain orientations oriented in substantial part transversely to the orientation of the first set of grains in the tibial plateau 110. These tubes provide for improved bending strength along the length of the stem 120 (within any or all of the stem body 123, gussets 125, and posterior fin 127), while the bending strength of the tibial plateau 110 benefits from the medial-lateral grain orientations resulting from the first portion additive manufacturing grain orientations describe herein.

As shown in FIGS. 7 and 7A, and as previously mentioned, the second portion additively manufactured may be the perimeter portion 115 around the tibial plateau 110 of the tibial component 100. As shown, build pathways 116 show the direction melting is accomplished in the perimeter portion 115 to cause the second set of grains to be oriented at least in substantial part transversely to the orientation of the first set of grains of the tibial plateau 110. The perimeter hoop formed by the perimeter portion 115 provides for improved stiffness along the edges of the tibial plateau 110, while the bending strength of the tibial plateau 110 benefits from the medial-lateral grain orientations resulting from the first portion additive manufacturing grain orientations describe herein.

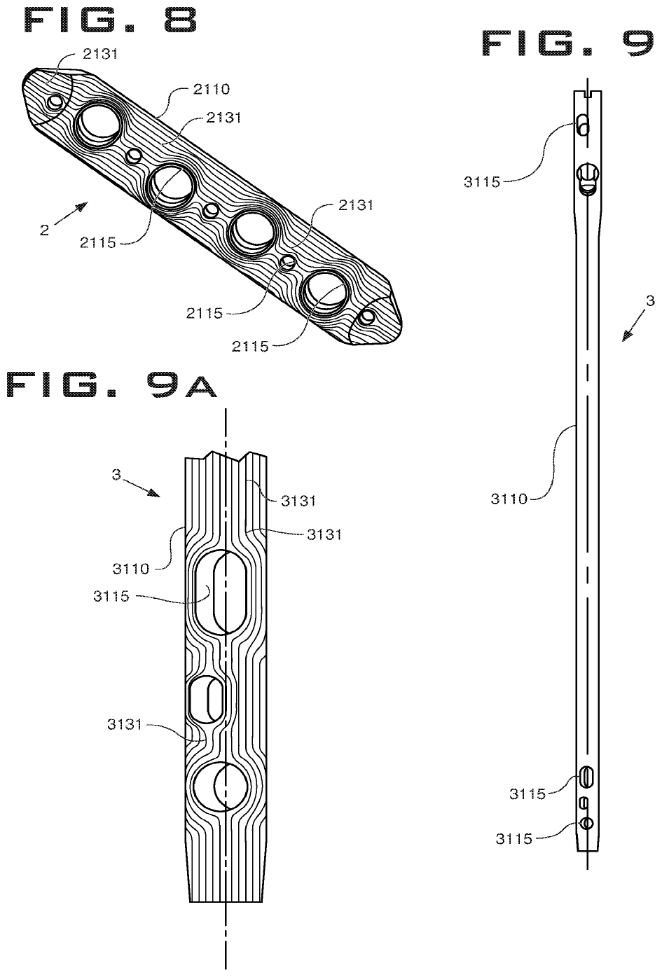

As previously mentioned, the medical implant manufactured or formed by the innovative methodology described herein can be any suitable implant now known or hereafter developed. For example, referring to FIG. 8, the medical implant may be a bone plate 2. Referring to FIGS. 9 and 9A, the medical implant may be an intramedullary device 3. The bone plate 2 and the intramedullary nails 3 may have any shape, configuration, etc. now known or hereafter developed. For example, some embodiments of the bone plate 2 and the intramedullary device 3 may include fixation screws and pins as part of the devices. As shown in FIG. 8, the bone plate 2 includes a body 2110 and multiple openings 2115 configured to receive pins or screws. As shown in FIGS. 9 and 9A, the intramedullary device 3 includes a body 3110 and multiple openings 3115 configured to receive pins or screws. Any other types of bone plates or intramedullary devices are contemplated to be within the scope of the disclosure.

In accordance with aspects of the present disclosure, as shown in FIG. 8, the first portion of the bone plate 2 may be embodied in the body 2110 additively manufacture by melting a granulized material along a build plane of the body 2110, as most clearly shown by the build pathways 2131. The granulized material may be any suitable material now known or hereafter developed including, for example, a titanium alloy, a steel alloy, another metal, a polymer, or any effective combination of granulized materials. The illustrated build pathways 2131 are not to scale and are intended to primarily show a general direction primarily along the longest dimension of the bone plate 2. Thus arranged, as shown, the build pathways 2131 may extend substantially parallel to a longitudinal axis of the bone plate 2. In other embodiments, build pathways of a bone plate could be transverse to the longest dimension of the bone plate 2 (e.g., transverse or angled relative to the longitudinal axis of the bone plate 2). In the illustrated embodiment, melting is accomplished with a series of substantially overlapping melting events as shown and described herein in association with FIGS. 4 and 4A. This act of melting orients a first set of grains of the material after cooling along the build plane of the first portion. For example, in one embodiment, the laser beam light is moved across the bone plate 2, parallel to the longitudinal axis of the bone plate 2, in a reciprocating direction to form the illustrated build pathways 2131.

In accordance with aspects of the present disclosure, as shown in FIGS. 9 and 9A, the first portion of the intramedullary device 3 may be embodied in the body 3110 additively manufacture by melting a granulized material along a build plane of the body 3110, as most clearly seen by the build pathways 3131. The granulized material may be any suitable material now known or hereafter developed including, for example, a titanium alloy, a steel alloy, another metal, a polymer, or any effective combination of granulized materials. The illustrated build pathways 3131 are not to scale and are intended to primarily show a general direction primarily along the longest dimension of the intramedullary device 3. Thus arranged, as shown, the build pathways 3131 may extend substantially parallel to a longitudinal axis of the intramedullary nail 3. In other embodiments, build pathways of an intramedullary device could be transverse to the longest dimension of the intramedullary device 3 (e.g., transverse or angled relative to the longitudinal axis of the intramedullary nail 3). In the illustrated embodiment, melting is accomplished with a series of substantially overlapping melting events as shown and described herein in association with FIGS. 4 and 4A. This act of melting orients a first set of grains of the material after cooling along the build plane of the first portion. For example, in one embodiment, the laser beam light is moved across the intramedullary nail 3, parallel to the longitudinal axis of the intramedullary nail 3, in a reciprocating direction to form the illustrated build pathways 3131.

In accordance with aspects of the present disclosure, the bone plate 2 and the intramedullary device 3 each include a second portion additively manufactured at least in part to the first portion by melting the granulized material to orient a second set of grains of the material after cooling at least in substantial part transversely to the orientation of the first set of grains. For example, in the case of the bone plate 2, the second portion may be one or more of an area surrounding the one or more openings 2115 or the perimeters of the one or more of the openings 2115 formed in the bone plate 2, which may also include at least part of an interior surface of the openings 2115. In the case of the intramedullary device 3, the second portion may be one or more of the area surrounding the one or more openings 3115 or perimeters of one or more of the openings 3115 formed in the intramedullary nail 3, which may also include at least part of an interior surface of the openings 3115. The second portion may also be referred to herein as a transverse portion having grains oriented transversely to the typical or prevailing orientation of the build pathway along the build plane of the bone plate 2 or the intramedullary device 3. The grain orientations around the area or perimeters of one or more of the openings 2115, 3115 provide a diversion of stress risers around the opening in combination with the bending strength advantages of the grain orientations resulting from the first portions additive manufacturing grain orientations along the longest dimensions of the bone plate 2 and intramedullary device 3.

Method embodiments of the invention include forming a medical implant by heating a first portion of a granulized material by irradiating the first portion of the granulized material along a first build pathway, such as for example, the build pathways 131 described in association with the tibial component 100, the build pathways 2131 described in association with the bone plate 2, and the build pathways 3131 described in association with the intramedullary device 3. The irradiation of the first portion melts the material such that grains of the material are oriented along the first build pathway 131, 2131, 3131 after the melted material cools. The first build pathway may be a series of substantially overlapping melting events as described herein in association with FIGS. 4 and 4A in the directions, with the equipment, and at the intensities describe or as are otherwise effective.

Method embodiments may also include heating a second portion of the granulized material by irradiating the second portion of the granulized material along a second build pathway, such as for example, the build pathways 116, 126 described in association with the tibial component 100, the build pathways about a perimeter of one or more of the openings 2115 in the bone plate 2, and the build pathways about a perimeter of the one or more of the openings 3115 in the intramedullary device 3. The irradiation of the second portion melts the material such that grains of the material are oriented along the second build pathway after the melted material cools. The second build pathway may be a series of substantially overlapping melting events as described herein in association with FIGS. 4 and 4A at least in substantial part transversely to the orientation prevailing direction of the first build pathway. The melting events may be in the directions, with the equipment, and at the intensities describe herein or as are otherwise effective.

Various embodiments of the devices described herein in whole or their components individually may be made from any biocompatible material. For example and without limitation, biocompatible materials may include in whole or in part: non-reinforced polymers, reinforced polymers, metals, ceramics, adhesives, reinforced adhesives, and combinations of these materials. Reinforcing of polymers may be accomplished with carbon, metal, or glass or any other effective material. Examples of biocompatible polymer materials include polyamide base resins, polyethylene, low density polyethylene, polymethylmethacrylate (PMMA), polyetheretherketone (PEEK), polyetherketoneketone (PEKK), a polymeric hydroxyethylmethacrylate (PHEMA), and polyurethane, any of which may be reinforced. Example biocompatible metals include stainless steel and other steel alloys, cobalt chrome alloys, zirconium, oxidized zirconium, tantalum, titanium, titanium alloys, titanium-nickel alloys such as Nitinol and other superelastic or shape-memory metal alloys.

Terms such as anterior, posterior, medial, lateral, proximal, distal, and the like have been used relatively herein. However, such terms are not limited to specific coordinate orientations, distances, or sizes, but are used to describe relative positions referencing particular embodiments. Such terms are not generally limiting to the scope of the claims made herein. Any embodiment or feature of any section, portion, or any other component shown or particularly described in relation to various embodiments of similar sections, portions, or components herein may be interchangeably applied to any other similar embodiment or feature shown or described herein.

While embodiments of the invention have been illustrated and described in detail in the disclosure, the disclosure is to be considered as illustrative and not restrictive in character. All changes and modifications that come within the spirit of the invention are to be considered within the scope of the disclosure.

While the present disclosure refers to certain embodiments, numerous modifications, alterations, and changes to the described embodiments are possible without departing from the sphere and scope of the present disclosure, as defined in the appended claim(s). Accordingly, it is intended that the present disclosure not be limited to the described embodiments, but that it has the full scope defined by the language of the following claims, and equivalents thereof. The discussion of any embodiment is meant only to be explanatory and is not intended to suggest that the scope of the disclosure, including the claims, is limited to these embodiments. In other words, while illustrative embodiments of the disclosure have been described in detail herein, it is to be understood that the inventive concepts may be otherwise variously embodied and employed, and that the appended claims are intended to be construed to include such variations, except as limited by the prior art.

The foregoing discussion has been presented for purposes of illustration and description and is not intended to limit the disclosure to the form or forms disclosed herein. For example, various features of the disclosure are grouped together in one or more embodiments or configurations for the purpose of streamlining the disclosure. However, it should be understood that various features of the certain embodiments or configurations of the disclosure may be combined in alternate embodiments, or configurations.

As used herein, an element or step recited in the singular and proceeded with the word “a” or “an” should be understood as not excluding plural elements or steps, unless such exclusion is explicitly recited. Furthermore, references to “one embodiment” of the present disclosure are not intended to be interpreted as excluding the existence of additional embodiments that also incorporate the recited features.

The use of “including,” “comprising,” or “having” and variations thereof herein is meant to encompass the items listed thereafter and equivalents thereof as well as additional items. Accordingly, the terms “including,” “comprising,” or “having” and variations thereof are open-ended expressions and can be used interchangeably herein. The phrases “at least one”, “one or more”, and “and/or”, as used herein, are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. All rotational references describe relative movement between the various elements. Connection references (e.g., engaged, attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority but are used to distinguish one feature from another. The drawings are for purposes of illustration only and the dimensions, positions, order and relative sizes reflected in the drawings attached hereto may vary.

1. A method of forming an orthopedic medical implant, the method comprising:
heating a first portion of a granulized material by irradiating the first portion of the granulized material along a first build pathway to melt the material such that grains of the material are oriented along the first build pathway after the melted material cools, wherein the first build pathway is a series of substantially overlapping melting events; and
heating a second portion of the granulized material by irradiating the second portion of the granulized material along a second build pathway to melt the material such that grains of the material are oriented along the second build pathway after the melted material cools, wherein the second build pathway is a series of substantially overlapping melting events that are oriented at least in part transversely to the orientation prevailing direction of the build pathway of the first portion;
wherein the act of heating a first portion of the granulized material by irradiating the first portion of the granulized material includes irradiating with a laser light beam such that a part of the first portion larger than a diameter of the laser light beam is molten while the first portion is being formed and adjacent areas overlap with one another primarily linearly along the build pathway.

2. The method of claim 1, wherein the first build pathway is a series of substantially overlapping melting events in a circular pattern.

3. The method of claim 2, wherein the second build pathway is a series of substantially overlapping melting events in a circular pattern.

4. The method of claim 1, wherein heating a first portion of a granulized material is performed by application of consistent heat and speed to the first portion of the granulized material.

5. The method of claim 1, wherein heating the first portion and second portion is performed by application variable heat to the first and second portions, respectively.

6. The method of claim 5, wherein the application of variable heat to the first and second portions is sufficient to polarize the material along the first build pathway of sufficient gradient depth and width to polarize the material.

7. The method of claim 1, wherein the medical implant is a knee arthroplasty implant including a tibial component including a tibial plateau and a stem portion, the first portion comprising the tibial plateau, the second portion comprising the stem portion.

8. The method of claim 7, wherein the tibial plateau further comprises a perimeter portion, the perimeter portion being formed by:
heating the perimeter portion of the granulized material by irradiating the perimeter portion of along a perimeter build pathway to melt the material such that grains of the material are oriented along the perimeter build pathway after the melted material cools, wherein the perimeter build pathway is a series of substantially overlapping melting events that are oriented at least in part transversely to the orientation prevailing direction of the build pathway of the tibial pathway.

9. The method of claim 1, wherein the medical implant includes a body portion having one or more openings formed therein, the first portion comprising the body portion, the second portion comprising an area surrounding the one or more openings.

10. The method of claim 1, wherein the medical implant is a bone plate including a body and one or more openings formed therein, the first portion comprising the body of the bone plate, the second portion comprising an area surrounding the one or more openings.

11. The method of claim 10, wherein the body of the bone plate includes a longitudinal axis, the first build pathway being orientated along the longitudinal axis of the bone plate.

12. The method of claim 1, wherein the medical implant is an intramedullary nail including a body and one or more openings formed therein, the first portion comprising the body of the intramedullary nail, the second portion comprising an area surrounding the one or more openings.

13. The method of claim 12, wherein the body of the intramedullary nail includes a longitudinal axis, the first build pathway being orientated along the longitudinal axis of the intramedullary nail.

14. The method of claim 1, wherein the granulized material is selected from one of a titanium alloy, a steel alloy, a metal, a polymer, or any effective combination of granulized materials.
